(19) United States
(12) Patent Application Publication
(10) Pub. No.: US 2024/0325701 A1
(43) Pub. Date: Oct. 3, 2024

(54) METHOD OF PRODUCING A MICRONEEDLE ARRAY, MICRONEEDLE ARRAY AND USE THEREOF

(71) Applicant: Cipherx Technologies LTD., London, Greater London (GB)

(72) Inventor: Ferdinand Kohle, London, Greater London (GB)

(21) Appl. No.: 18/573,950

(22) PCT Filed: Jun. 27, 2022
(86) PCT No.: PCT/GB2022/051643

§ 371 Date: Dec. 22, 2023

(30) Foreign Application Priority Data
Jun. 25, 2021 (GB) 2109228.3

(51) Int. Cl. A61M 37/00

(52) U.S. Cl. CPC A61M 37/0015; A61M 37/0084; A61M 2037/0053

(57) ABSTRACT

Disclosed is a method of forming a microneedle array having a predefined microneedle pattern. The method comprises transforming digital information in a digital input pattern/image into a desired microneedle pattern, such that locations of microneedles in the microneedle pattern correspond to pixels in at least a portion of the digital input pattern or image; and forming a microneedle array having the desired microneedle pattern. Also disclosed is a method of forming a microneedle array having the predefined microneedle pattern using a master microneedle mold. The method comprises masking one or more holes in the master mold at predefined locations to create an array of unmasked holes corresponding to a desired microneedle pattern; and forming a microneedle array having the desired microneedle pattern using the masked master mold.

### TECHNICAL FIELD

This invention relates generally to a microneedle array and method of producing a microneedle array using a molding process, particularly, but not exclusively, microneedle arrays suitable for delivering a tattoo.

### BACKGROUND TO THE INVENTION

Microneedles are microscopic needles that can be used to deliver a wide range of substances such as drugs, vaccines, hormones, dyes and pigments, nano- and microparticles, etc. across various barriers such as skin or tissue. They have attracted increasing scientific and industrial interests in the past two decades due to their advantages over traditional hypodermic needles including painless penetration, low cost, excellent therapeutic efficacy, reduced risk of infections, and relative safety.

In particular, microneedle patches that comprise a microneedle array on a flexible adhesive substrate that can be self-administered and/or applied directly to a subject's skin are being developed for transdermal delivery of a cargo material. These microneedles can be made in various geometries from a range of materials including metals, polymers and semiconductors, and employ a range of delivery strategies. Solid microneedles (e.g. metal) puncture the tissue prior to application of a drug-loaded patch or are pre-coated with the cargo prior to insertion. Hollow bore microneedles allow diffusion or pressure driven flow of cargo through a central passage into the skin/tissue. Dissolvable microneedles made from non-toxic biodegradable polymers that contain or encapsulate the cargo release their cargo into the skin/tissue over time by dissolving or degrading upon insertion. Dissolving polymer microneedles are particularly advantageous due to the low cost or materials and relative simple manufacture process that is well suited to mass production. Polymer microneedle arrays are typically formed by casting one or more solutions containing the required cargo into a master mold comprising an array of holes or cavities in a given geometry and arrangement, solidifying the solution, and releasing the array from the mold. In this way, microneedle arrays can be reliably and repeatedly formed in various geometries using the same reusable master mold.

In addition to or instead of drug delivery, microneedles can be used to deliver molecules, dyes, pigments, nanoparticles or microparticles (hereby referred to as “inks”) into the skin to create permanent, semi-permanent or temporary tattoos which can be purely aesthetic, decorative or functional e.g. containing text information. Such microneedles arrays are known as tattoo patches. However, tattoo patches require a microneedle array that is either configured with a specific needle pattern based on a desired image, or configured to deliver cargo from a specific subset of needles in the array based on the desired image. As the image may in principle be arbitrary, scalable and cost-effective production of customised tattoo patches poses a non-trivial technical problem. Existing approaches to producing tattoo patches typically suffer from complex preparation and fabrication processes and/or lack the versatility and capability of changing the image design quickly.

Examples of known tattoo patches include US2020/330740A1 and US2019/015650A1. US2019/015650A1 discloses a microneedle patch comprising dissolvable polymer microneedles encapsulating the cargo to create a tattoo, or deliver a bio-active agent in combination with a tattoo to create a record of the administration on the skin that can be imaged at a later time. In this case, microneedle patches with the desired microneedle pattern are formed by solution casting using a master mold specifically designed for the particular tattoo/image. US2020/330740A1 discloses a customisable microneedle substrate for printing a multicolor image/tattoo on the skin. The microneedle substrate comprises an array of individual movable microneedle blocks held together under compression to form a desired image, each block including one or more rigid metal microneedles in a desired pattern and where each microneedle constitutes a pixel of the image. In this case, microneedles are coated with inks in predefined patterns by electrostatically charging specific microneedles in the array and using an oppositely charged ink roller to transfer ink to the charged needles.

There is therefore a need for an improved versatile, scalable and cost effective method of producing microneedle arrays with a predefined microneedle pattern for targeted delivery of cargo. Aspects and embodiments of the present invention have been devised with the foregoing in mind.

### SUMMARY OF THE INVENTION

According to a first aspect of the invention, there is provided a method of forming a microneedle array. The microneedle array may be formed by a molding process. The method may use a (female) master microneedle mold with an array of holes for forming a microneedle array. The array of holes may be defined by an array geometry i.e. relative location of holes, spacing/separation. Each hole defines the shape and size of a microneedle. The method may comprise masking one or more holes in the master mold at predefined locations to create an array of unmasked holes. The array of unmasked holes may correspond to a desired microneedle pattern or at least a portion of a desired microneedle pattern. The method may further comprise forming a microneedle array having the desired microneedle pattern using the masked master mold. The microneedle array may be formed from or comprise one or more non-toxic and biodegradable polymers or polymer solutions. Each microneedle may contain a cargo, such as an ink, drug, vaccine, hormone, nanoparticle, microparticle or any other substance/material to be delivered into the skin or tissue.

In this context, a microneedle cargo refers to a specific substance/material or combination of substances/materials to be delivered by the microneedle. A suitable cargo may include, but is not limited to, any one or more of: fluorescent and non-fluorescent dyes (e.g. cyanine dyes, carborhodamine dyes, BODIPY dyes, xanthene dyes, eosins, and rhodamines, methylene blue), pigments (carbon), paramagnetic and diamagnetic molecules, drugs, peptides, enzymes, hormones, sugars, lipids, nucleases, vaccines, biosensing materials (e.g. glucose-responsive fluorescent microbeads for glucose monitoring, seminaphtorhodafluor (SNARF) or anthocyanin encapsulating microparticles for pH value determination, fluorescent diaza-15-crown-5 (Sodium green) for sodium level determination), inorganic nanocrystals (e.g. near infrared emitting (NIR) copper quantum dots), nanoparticles (e.g. polyethylene glycol (PEG) stabilized silica nanoparticles encapsulating dyes and/or other functional moieties also known as Cornell Dots, metal oxide nanoparticles, etc.), microbeads or polymeric microparticles (poly (methyl methacrylate) (PMMA) microparticles encapsulating dyes and/or other functional moieties, microparticles that slowly degrade over time in the skin, etc.), cosmetic active agents and nutrients.

Herein, an “ink” refers to one or more of molecules, visible/invisible dyes, pigments, fluorescent dyes, nanoparticles or microparticles. The microneedle array may be or comprise a tattoo patch.

The invention provides a method of making microneedle arrays with various different patterns using the same standard master mold. The desired needle pattern can be generated from an arbitrary image/pattern using a computer program and the graphical information is translated into a microneedle arrangement using the reusable master mold and one or more sacrificial or reusable masking layers that are customised based on the desired pattern. Each needle can be can be made from the same or different polymer solution and contain the same or different cargo. The process can therefore be used to make one-time use microneedle patches as a means to simultaneously deliver multiple different materials to precise locations relative to each other in the skin/tissue, e.g. to imprint visible or invisible multicolor tattoos and/or multiple prophylactic, therapeutic, diagnostic or cosmetic materials into the skin/tissue. The method therefore provides an improved versatile, scalable and cost effective method of producing microneedle arrays with a predefined microneedle pattern for targeted delivery of cargos.

Each microneedle in the array may have a relative position defined according a digital input pattern. The desired microneedle pattern may be based on at least a portion of a digital input pattern or image, and the locations of un-masked holes in the master mold may correspond to pixels of the digital input pattern or image. The input image or pattern may be or comprise a machine readable optical indicia encoding information such as a barcode or quick response (QR) code, text and/or an image. The desired microneedle pattern may be unique. The digital input image and microneedle pattern may comprise or encode a unique identifier (ID). In one example application, a tattoo patch formed according to the present invention can be used to transfer a unique identifier tattoo into/onto the skin of a person, which may be visible or invisible (under visible light). The ID tattoo can be associated with conventional tattoo or other form of body art and used as a means to authenticate the tattoo or body art. For example, the ID tattoo can be read or imaged by an imaging device, such as a camera (visible or infrared) and the detected ID tattoo can be compared against digital input images stored in a database to determine if there is a match. Digital input images in the database can be associated with a person or artist, and a match would indicate the tattoo or body is authentic and associated with the particular artist.

The step of masking one or more holes in the master mold may comprise aligning a masking layer over the master mold to cover the one or more holes in the master mold at the predefined locations. The aligned masking layer may be attached to the master mold.

The masking layer may comprise a plurality of openings that correspond to the desired microneedle pattern and geometry of the hole array. In this way, the openings of the masking layer can be aligned to the hole array to mask one or more holes in the master mold and create an array of unmasked holes at locations corresponding to the desired needle pattern.

The method may comprise determining a desired microneedle pattern based on a digital input pattern/image and the geometry of the array of holes of the master mold. The needle pattern may be determined such that the locations of un-masked holes in the master mold correspond to pixels of the digital input pattern or image.

The method may comprise determining locations of one or more holes in the master mold to be masked based on a digital input pattern/image and the geometry of the array of holes of the master mold. The needle pattern may be determined such that the locations of un-masked holes in the master mold correspond to pixels of the digital input pattern or image.

Each pixel in the digital input pattern/image may be represented by one or more microneedles in the desired microneedle pattern.

The method may further comprise generating a microneedle image file defining the microneedle array properties based at least in part on a digital input pattern/image and the geometry of the array of holes of the master mold. Each pixel of the microneedle image file may correspond to one or more microneedles of the desired microneedle pattern. Each pixel of the microneedle image file may contain microneedle information including one or more of: microneedle location, microneedle material, microneedle geometry, microneedle number, microneedle additives, microneedle cargo, and relative amount of cargo. The locations of the one or more holes in the master mold to be masked may be defined in the microneedle image file.

The method may comprise forming a masking layer configured to cover the one or more holes in the master mold at the predefined locations based on a predefined needle pattern. Suitable materials for the masking layer include, but are not limited to: polyethylene (PE), polyethylene terephthalate (PET), polypropylene (PP), polystyrene, polylactic acid and others. The masking layer may have a thickness in the range of approximately 10 μm to 500 μm.

The masking layer may be formed by customizing a standard masking layer or forming a customised masking layer based on the desired pattern/digital input pattern/microneedle image file.

The (standardised) masking layer comprises an array of openings corresponding to or matching the array or holes or the locations of holes in the master mold. In this case, forming the masking layer may comprise selectively closing, filling or covering one or more openings in the standardised masking layer to create an array of openings corresponding to the desired microneedle pattern. Closing the one or more openings may comprise casting a photopolymer over the (standard) masking layer and exposing the photopolymer at the locations of the one or more openings to be closed. Residual photopolymer may be removed or washed away.

The one or more openings may be closed using a photo-induced photo-polymerization process such as a laser-induced photo-polymerisation process or a photolithography process. The photo-polymerisation process may be based on microneedle location information in the microneedle image file.

Alternatively, the step of forming the masking layer may comprise forming one or more openings in the masking layer at predefined locations corresponding to the desired microneedle pattern. This may be achieved by a photolithographic process, or laser drilling process, or mechanical drilling process (e.g. based on microneedle location information in the microneedle image file).

Forming the microneedle array may comprise casting a polymer solution into the masked master mold. Forming the microneedle array may further comprise curing or solidifying the polymer solution. The polymer solution may contain or encapsulate a cargo to be delivered upon insertion of the microneedle array to the skin/tissue. The polymer solution may comprise one or more additives, e.g. to control the rate of dissolution of the microneedle upon insertion, as is known in the art. The cargo may be dispersed in the solution or bonded to the polymer molecules.

Alternatively or additionally, cargo or a polymer solution containing a cargo may be deposited into the unmasked holes of the master mold prior to solution casting, e.g. based on information in the microneedle image file. The cargo or polymer solution containing a cargo may be deposited by inkjet printing.

One or more microneedles in the microneedle array may comprise a different cargo to at least one other microneedle in the microneedle array.

Inkjet printing may allow multiple different cargos to be deposited into the array of unmasked holes in the same step. In this way, the process may require a single masking layer with the entire microneedle pattern to produce a multi-colour tattoo and/or multi-cargo microneedle array.

Otherwise, where the polymer solution contains the cargo, multiple masking steps can be used to produce a multi-colour tattoo and/or multi-cargo microneedle array. In this case, the step of masking one or more holes (i.e. a first subset of holes) in the master mold creates a first array of unmasked holes corresponding to a first portion of the desired microneedle pattern, and the step of forming the microneedle array forms a first portion of a microneedle array having the first portion of the desired microneedle pattern using the masked master mold and a polymer solution containing a first cargo.

Suitable materials for the polymer microneedles include polyvinyl alcohol (PVA), hyaluronic acid, polyvinyl pyrrolidone (PVP), sugar based materials, polyethylene glycol, poly(D,L-lactide) (PLA), poly (methyl vinyl ether-co-maleic acid) (PMVEMA), carboxymethyl cellulose (CMC), hydroxy acids), and any other suitable materials, as is known in the art.

The method may then further comprise masking one or more different holes (i.e. a second subset of holes) in the master mold to create a second array of unmasked holes corresponding to a second portion of the desired microneedle pattern, and forming a second portion of the microneedle array having the second portion of the desired microneedle pattern using the re-masked master mold and a polymer solution containing a second cargo.

The masking steps may comprise aligning a first masking layer having a first array of openings over the master mold to create a first array of unmasked holes, and after forming the first portion of the microneedle array, removing the first masking layer and aligning a second masking layer having a second array of openings over the master mold to create the second array of unmasked holes.

This processing can be repeated multiple times as necessary for the design of the tattoo or microneedle array.

The method may further comprise forming a substrate layer over the microneedle array. The substrate may be substantially flexible and comprise an adhesive layer or portion to form a microneedle patch.

The method may further comprise releasing the microneedle array from the masked master mold.

According to a second aspect of the invention, there is provided a microneedle array formed by the method of the first aspect.

According to a third aspect of the invention, there is provided a method of forming a microneedle array. The method comprises transforming digital information in a digital input pattern/image into a desired microneedle pattern, such that locations of microneedles in the microneedle pattern correspond to pixels in at least a portion of the digital input pattern or image. The digital information may be transformed into microneedle data defining the desired microneedle pattern. The method may further comprise forming a microneedle array having or according to the desired microneedle pattern. Forming the microneedle array may comprise forming the microneedle array based on the microneedle data.

Forming the microneedle array may comprise a molding process, or a three-dimensional printing process. The molding process may be the process of the first aspect. Alternatively, three-dimensional printing may be used to make a male master mold for a desired microneedle pattern.

The step of transforming is computer implemented, i.e. using a computing/processing device. The step of transforming may comprise generating a microneedle image file defining the properties of the microneedle array based at least in part on the digital input pattern/image. The microneedle image file may be or comprise the microneedle data. Each pixel of the microneedle image file may correspond to one or more microneedles of the desired microneedle pattern. The microneedle pattern may be defined by pixels in the microneedle image file. The location of pixel of the microneedle image file may correspond to the location a microneedle or group of microneedles in the desired microneedle pattern. Forming the microneedle array having or according to the desired microneedle pattern may comprise forming the microneedle array based on information in the microneedle image file.

Each pixel of the microneedle image file may contain microneedle information. The microneedle information may include one or more of: microneedle location, microneedle material, microneedle geometry, microneedle number, microneedle cargo, and relative amount of cargo. Each pixel may have a plurality of associated pixel values defining a property of the microarray at that pixel location. The pixel may comprise binary pixel value indicating the presence of a microneedle (e.g. 1 for microneedle and 0 for no microneedle). The pixel may comprise a value or indicating the cargo and/or relative amount of cargo.

The step of generating a microneedle image file may comprise detecting an image feature in the digital input pattern/image, and generating a microneedle pattern based at least in part on the detected image feature. The shape, area and/or perimeter of the microneedle pattern (i.e. the spatial extent of the pattern) may correspond to the shape, size/area, and/or perimeter of the image feature.

Each microneedle may contain a cargo to be delivered into the skin or tissue. Where the cargo is an ink or pigment, the step of generating a microneedle image file may further comprise determining a colour of the ink or pigment for each microneedle in the microneedle pattern based on a colour of one or more pixels of the image feature, or based on the colour of one or more pixels in a region surrounding the image feature. Optionally the colour of the cargo of the microneedles may be determined so as to substantially match the colour of one or more pixels in or around the image feature. In this way, the microneedle array may be used to mask or camouflage a feature on the skin, such as a variation of pigmentation or a scar.

The digital input image may be or comprise an image of an area of skin or tissue including a pigmentation feature or a scar feature. In this case, the area and/or perimeter of the desired microneedle pattern may correspond to the size and shape of the pigmentation or scar feature.

The method may comprise generating a microneedle image file defining a microneedle array configured to mask or camouflage a pigmentation feature or a scar feature in an area of skin or tissue.

According to a fourth aspect of the invention, there is provided a microneedle array for delivering cargo material into the skin or tissue. The microneedle array comprises microneedles formed in a desired microneedle pattern. The desired microneedle pattern is based on at least a portion of a digital input pattern or image, and the locations of the microneedles in the microneedle pattern correspond to pixels in the at least a portion of the digital input pattern or image.

The microneedle pattern may be configured to convey graphical information.

The shape, area and/or perimeter of the microneedle pattern (i.e. the spatial extent of the pattern) may correspond to the shape, size/area, and/or perimeter of an image feature in the input image.

The digital input image may be or comprise an image of an area of skin or tissue including a pigmentation feature or a scar feature. In this case, the area and/or perimeter of the desired microneedle pattern may correspond to the size and shape of the pigmentation or scar feature.

Each microneedle contains a cargo to be delivered into the skin or tissue. The cargo may comprise one or more of: an ink/pigment, a prophylactic drug, a therapeutic drug, a diagnostic or biosensing material, and a cosmetic material.

Where the cargo is an ink or pigment, the colour of the ink or pigment for each microneedle in the microneedle pattern may be based on a colour of one or more pixels of the image feature, or based on the colour of one or more pixels in a region surrounding the image feature. Optionally the colour of the cargo of the microneedles may be determined so as to substantially match the colour of one or more pixels in or around the image feature. In this way, the microneedle array may be used to mask or camouflage a feature on the skin, such as a variation of pigmentation or a scar.

The digital input image may be or comprise an image of an area of skin or tissue including a pigmentation feature or a scar feature. The microneedle array may be configured to camouflage the pigmentation feature or scar feature.

The microneedle array may comprise one or more microneedles comprising a first cargo to be delivered; and one or more microneedles a second cargo to be delivered, the second cargo being different to the first cargo.

The microneedle array may be formed by a molding process or a three-dimensional printing process.

The microneedle array is formed using the method of the first or third aspect. A three-dimensional printing process may be used to make a male master mold for a desired microneedle pattern.

The microneedle array may define a needle pattern configured to convey graphical information. One or more microneedles may contain or encapsulate a first cargo to form a first portion of the needle pattern, and one or more other microneedles may contain or encapsulate a second cargo to form a second portion of the needle pattern.

The microneedle array may be configured to deliver a biosensing tattoo that conveys graphical information and changes colour or visibility in response to a change in body chemistry or other factors. Optionally preferably, the first cargo may comprise an ink, and the second cargo may comprise a bio-sensing material configured to change colour or visibility in response to an interaction with a chemical substance.

According to a fifth aspect of the invention, there is provided a microneedle for delivering multiple cargo materials into the skin or tissue. The array may comprise one or more microneedles formed from or comprising a polymer material containing or encapsulating a first cargo to be delivered. The array may comprise one or more microneedles formed from or comprising a polymer material containing or encapsulating a second cargo to be delivered, the second cargo being different to the first. The microneedle array may be formed by a molding process, such as the method of the first aspect.

The microneedle array may define a needle pattern configured to convey graphical information. The one or more microneedles containing or encapsulating the first cargo may form a first portion of the needle pattern, and the one or more microneedles containing or encapsulating the second cargo may form a first portion of the needle pattern.

The microneedle array may be configured to deliver a biosensing tattoo that conveys graphical information and changes colour or visibility in response to a change in body chemistry or temperature, or external factors such as temperature, light exposure, magnetic fields, or audio and electro-magnetic exposure and/or signals.

The first cargo may comprise an ink and/or a biosensing material. The second cargo may comprise a biosensing material or biosensing ink and/or an ink. A bio-sensing material may be defined as a material or substance configured to change colour or visibility in response to an interaction with a chemical substance. When deposited in the skin or tissue of a subject, the bio-sensing material may change colour or visibility in response to a change in body chemistry or temperature, or external factors such as temperature, light exposure, magnetic fields, or audio and electro-magnetic exposure and/or signals. For example, the biosensing material may be responsive to a pH value, saccharides (e.g. glucose), ions (e.g. sodium, potassium, calcium, magnesium, chloride, bicarbonate), lipids, nucleic acids, and proteins. Examples of biosensing materials include functionalised nano/microparticles.

In an alternative embodiment, microneedles in the biosensing microneedle array may be formed from or comprise a hydrogel containing or encapsulating a cargo. Hydrogels are biocompetable and highly tunable materials that swell after insertion instead of dissolving. In this case, after insertion and sensing the microneedle patch is removed and analyzed to determine the sensed parameter, for example with a smart phone, e.g. using a camera and/or other functionalities.

According to a sixth aspect of the invention, there is provided a microneedle array configured to deliver a biosensing tattoo that conveys graphical information in response to a change in a sensed parameter, e.g. body chemistry or temperature, or external factors such as temperature, light exposure, magnetic fields, or audio and electro-magnetic exposure and/or signals. The array may comprise one or more microneedles formed from or comprising a (first) polymer material containing or encapsulating a (first) cargo to be delivered. The cargo may comprise a bio-sensing material or biosensing ink configured to change colour or visibility in response to an interaction with a chemical substance.

The microneedle array may comprise one or more microneedles formed from or comprising a polymer material containing or encapsulating a second cargo to be delivered, the second cargo being different to the first. Optionally or preferably, the second cargo may comprise an ink. The first cargo may further comprise an ink.

According to a seventh aspect of the invention, there is provided a method of authenticating a first tattoo using a second tattoo comprising a machine readable optical indicia encoding authentication information. The method may comprise reading the authentication information encoded in the second tattoo. The method may comprise authenticating the first tattoo by comparing the authentication information to a database of identifiers associated with registered tattoo artists. The database may be stored at a remote server. The second tattoo may be formed using a tattoo patch produced using the method of the first aspect. The database of identifiers may comprise the input digital images/patterns used to create the tattoo patches.

According to an eighth aspect of the invention, there is provided a use of the microneedle array of any preceding aspect to apply a tattoo on the skin or tissue of a subject.

According to a ninth aspect of the invention, there is provided a use of the microneedle array of any preceding aspect to camouflage a skin pigment or scar feature on the skin or tissue of a subject.

According to a tenth aspect of the invention, there is provided a use of the microneedle array of any preceding aspect to apply a machine-readable optical indicia encoding information on the skin or tissue of a subject.

According to an eleventh aspect of the invention, there is provided a use of the microneedle array of any preceding aspect to authenticate a first tattoo of a subject, wherein the microneedle array is configured to deliver a second tattoo on the skin or tissue of the subject having the first tattoo comprising a machine-readable optical indicia encoding authentication information, and wherein the first tattoo is authenticated by reading the authentication information encoded in the second tattoo; and comparing the authentication information to a database of identifiers associated with registered tattoo artists.

Features which are described in the context of separate aspects and embodiments of the invention may be used together and/or be interchangeable. Similarly, where features are, for brevity, described in the context of a single embodiment, these may also be provided separately or in any suitable sub-combination. Features described in connection with the microneedle array(s) may have corresponding features definable with respect to the method(s), and vice versa, and these embodiments are specifically envisaged.

### BRIEF DESCRIPTION OF DRAWINGS

In order that the invention can be well understood, embodiments will now be discussed by way of example only with reference to the accompanying drawings, in which:

FIG. 1 shows block diagram of a method of producing a microneedle array according to an embodiment of the invention;

FIGS. 2(a) and 2(b) show schematic diagrams of an example microneedle array that can be produced using the method of FIG. 1;

FIGS. 3(a) and 3(b) show schematic diagrams of a transdermal delivery of cargo using the microneedle array of FIG. 2.

FIGS. 4(a) to 4(d) show schematic diagrams of female master molds with different hole patterns;

FIGS. 5(a) and 5(b) illustrate a single masking step process for producing a single cargo microneedle array according to an embodiment of the invention;

FIGS. 6(a) to 6(i) illustrate a multi-masking step process for producing a multi-cargo microneedle array according to an embodiment of the invention;

FIGS. 7(a) to 7(f) illustrate an inkjet printing process for producing a multi-cargo microneedle array according to another embodiment of the invention;

FIGS. 8(a) to 8(c) illustrate a process for forming a customised masking layer according to an embodiment of the invention;

FIG. 9 illustrates a process for translating graphical information into microneedle data;

FIGS. 10(a) and 10(b) show schematic diagrams of an example microneedle patch that can be produced using the method of FIG. 1;

FIG. 11 shows block diagram of a method of producing a microneedle array according to another embodiment of the invention;

FIG. 12 shows further steps of the method of FIG. 11; and

FIG. 13 shows a scanning electron micrograph of an example microneedle array.

It should be noted that the figures are diagrammatic and may not be drawn to scale. Relative dimensions and proportions of parts of these figures may have been shown exaggerated or reduced in size, for the sake of clarity and convenience in the drawings. The same reference signs are generally used to refer to corresponding or similar features in modified and/or different embodiments.

### DETAILED DESCRIPTION

Technological advances in nano- and microfabrication techniques allow us to re-think skin as a new interface for biosensing and data storage/encryption in the form of visible or invisible tattoos that can encode data (e.g. animal identification, blood type, vaccination history, tattoo artist signature, audio files, QR codes, etc.) in a graphic that can be read or imaged, and/or include bio-sensing materials that change colour or visibility in response to changes in the body's chemistry (e.g. pH value, saccharides (e.g. glucose), ions (e.g. sodium, potassium, calcium, magnesium, chloride, bicarbonate), lipids, nucleic acids, and proteins) to provide a tattoo with sensing functionality. To realise such applications of tattoos requires a means for easy and precise control of material deposition into the skin/tissue that is reproducible and compatible with a variety of materials (cargos), including visible pigments, invisible inks, fluorescent inks, biosensing materials, vaccines, therapeutic materials etc. Microneedle arrays or microneedle patches are an ideal candidate for transferring computer-generated information physically into the skin or tissue of a subject using a variety of different cargos including inks, drugs, biosensing materials etc. as previously described.

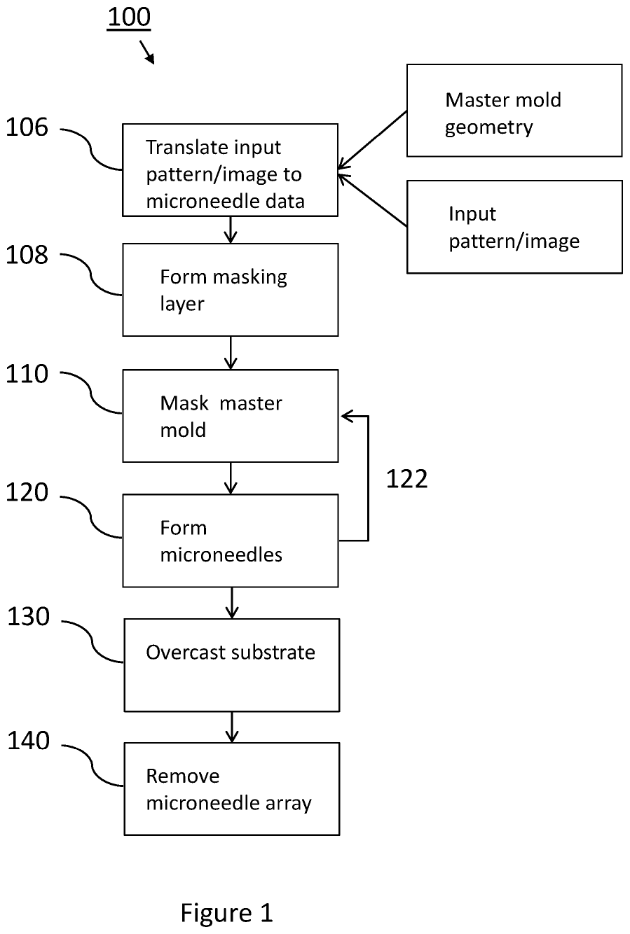

FIG. 1 shows a schematic block diagram of a method 100 of producing a microneedle array with a predefined needle and cargo pattern according to an embodiment of the invention.

The method 100 is based on the well-established micro-molding process in which a microneedle array is formed by casting a polymer solution containing a cargo into a reusable female master mold. However, in contrast to traditional microneedle fabrication techniques in which the microneedle pattern is determined entirely by the properties of the female master mold and the microneedle cargo is the same across the microneedle array, the method 100 utilises one or more novel masking steps to cover specific regions of the master mold and create a masked master mold that can be used to form microneedle arrays with customised microneedle and/or cargo patterns from the same master mold.

The method 100 therefore provides a simple, cost effective and scalable solution to producing microneedle arrays with customised needle and cargo patterns, whereby individual microneedles can be made to have predefined positions relative to each other and can be made from different materials that can deliver different cargos. Although embodiments are described below in the context of tattoo patches that deliver an ink cargo, the method is not limited to producing tattoo patches, and in principle any cargo suitable for delivery via a microneedle array can be used.

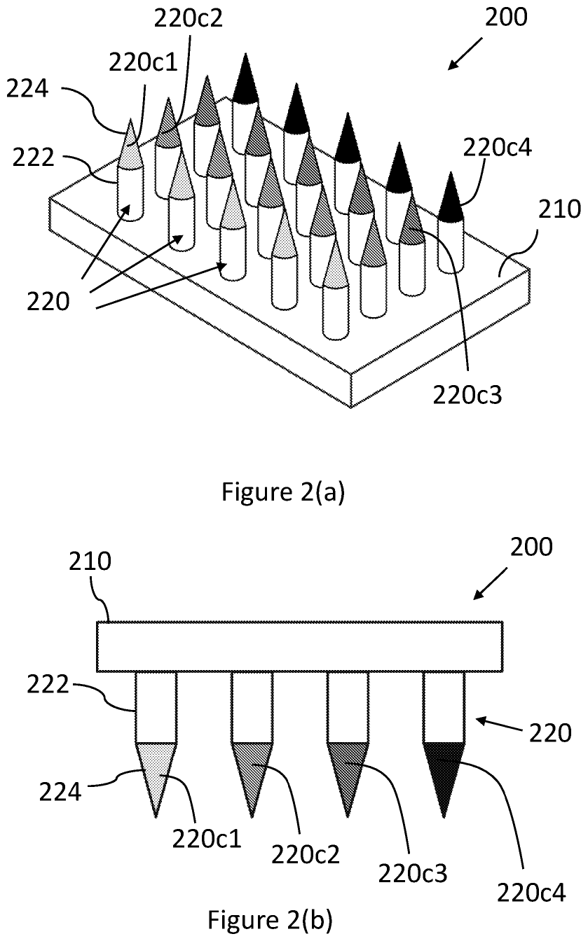

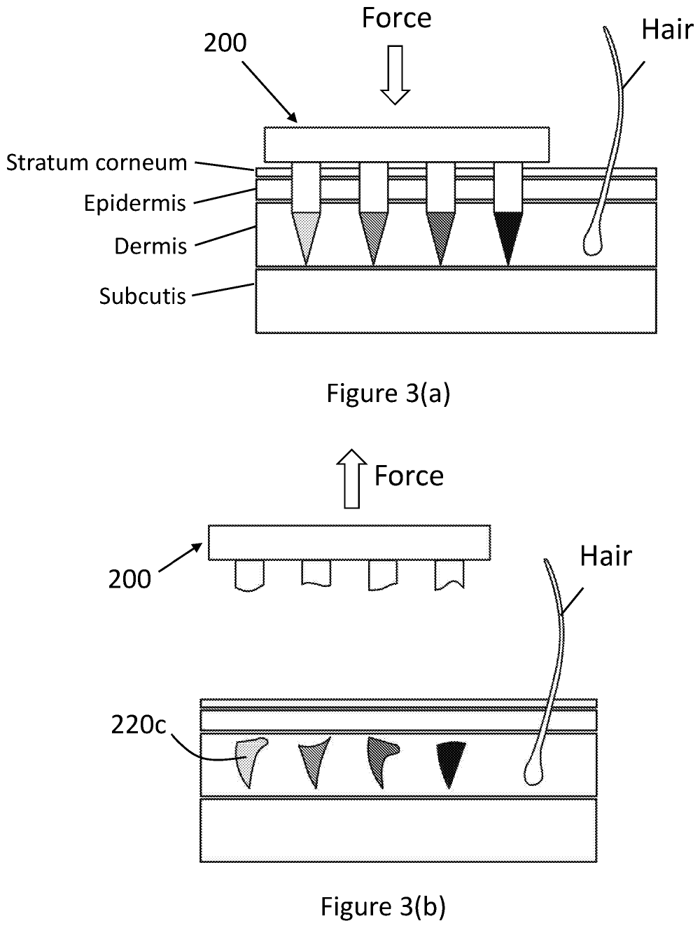

FIGS. 2(a) and 2(b) show schematic diagrams of an example multi-cargo microneedle array 200 that can be produced using the method 100. The microneedle array 200 comprises a substrate 210 and an array of microneedles 220 extending from or attached to the substrate 210 as shown. In the example shown, each microneedle 220 comprises a base portion 222 and a tip portion 224, however it will be appreciated that microneedles 200 can be formed with different geometries, and with geometries that vary across the array, depending on the design of the specific master mold. The microneedles 220 are formed from or comprise a biodegradable dissolvable polymer material that contains or encapsulates a cargo 220c to be delivered into the skin or tissue of a subject. In alternative embodiments, microneedles 200 can be formed from or comprise a hydrogel material that contains or encapsulates a cargo 220c to be delivered, but does not dissolve. Although the cargo 220c is shown located in the tip 224, in practice the cargo 220c can be also be located in the base 222 depending on the fabrication process, as will be described in more detail below. The polymer material is configured to dissolve within a short period of time after insertion into the skin or tissue to release the cargo 220c into the skin, typically within 1-30 minutes after insertion, as shown in FIGS. 3(a) and 3(b). The microneedle array 200 is applied by hand or by a mechanical actuator (not shown). Suitable materials for the dissolvable polymer microneedles 220 include polyvinyl alcohol (PVA) and hyaluronic acid, but they can also be made from a wide range of other suitable materials, as is known in the art (e.g. polyvinyl pyrrolidone (PVP), sugar based materials, polyethylene glycol, poly(D,L-lactide) (PLA), poly (methyl vinyl ether-co-maleic acid) (PMVEMA), carboxymethyl cellulose (CMC), hydroxy acids). One or more additives (e.g. sugars) can be included to modify and control the rate of dissolution, as is known in the art.

The cargo 220c can comprise visible or invisible inks, nanoparticles, microparticles, biosensing materials (e.g. functionalised nano/microparticles), hormones, enzymes, peptides, vaccines, cosmetic materials (such as nutrients, pigments) and/or others as described previously. Different microneedles 220 of the same array 200 can contain different cargos, combinations of cargos, or amount or concentrations of the same cargo.

The substrate 210 can be made from the same or different material as the microneedles 220. For example, a separate solution casting step can be used to form a substrate 210 from a different material to the microneedles 220, as described below. The substrate 210 is substantially flexible in order to accommodate curvature of skin at the region of application. A typical substrate 210 may have a width in the range 1 cm to 20 cm depending on the use/application or size of the tattoo. In a preferred embodiment, the substrate 210 is larger than the area of the microneedle array 200, and the area surrounding the array 200 contains an adhesive coating to provide a microneedle patch that can be secured to the skin after application (see FIG. 3).

The needle length determines the outcome of the cargo deposition. For example, with reference again to FIG. 3(a), human skin is comprised of several layers: the stratum corneum (approximately 10-30 μm thick), the epidermis (approximately 50-15 μm thick), and the dermis (approximately 600-2000 μm thick). For permanent ink tattoos, ink must be deposited within the deep dermis layer. If the deposition is not deep enough, ink will be shed with the skin over time, resulting in a semi-permanent or temporary tattoo.

In one example, to achieve a permanent black tattoo, microneedles 220 can be made of a hyaluronic acid polymer matrix embedding a carbon pigment. The length of the needles can be in the range 200-2500 μm, the radius of the base portion 222 can be in the range 50-500 μm, and the radius of the tip portion 224 in the range 1-20 μm, with a needle spacing in the range 200-1000 μm (although the minimum needle spacing is only limited by the master mold fabrication technique).

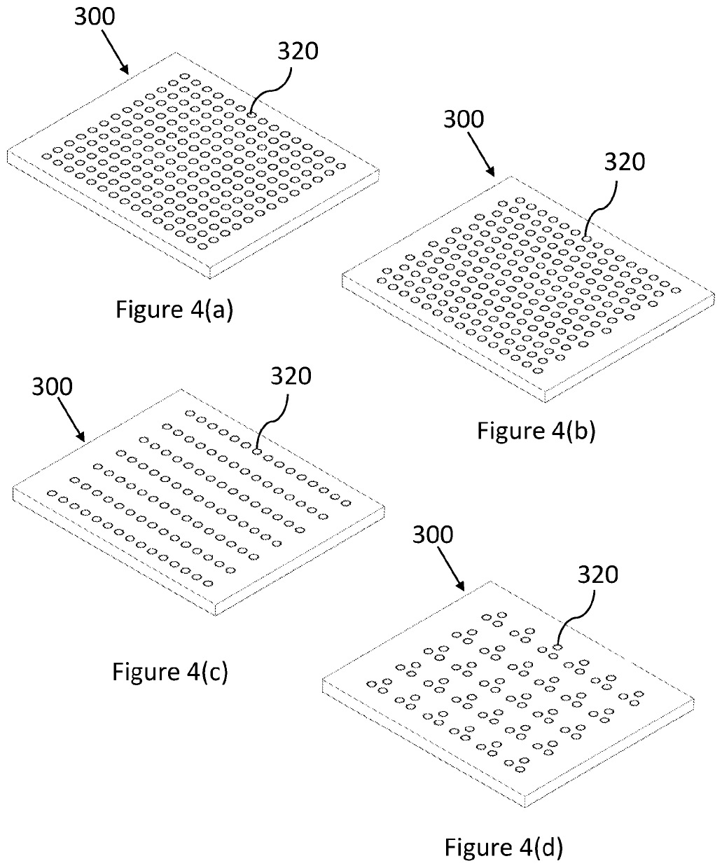

Master molds have an array of micro-holes or cavities that define the geometry of the individual microneedles (e.g. size and shape of each needle) and the needle-to-needle spacings. Master molds are typically made by casting silicone, polydimethylsiloxane (PDSM) or other suitable material over a male microneedle master mold that is produced by three-dimensional (3D) printing, two-photon polymerisation, photolithographic processes, or other suitable technique. Master molds are produced with a regular hole pattern such as a square array for common applications such as drug delivery, but the holes and the resulting microneedle array can in principle be made in any pattern according to the design of male master mold. FIGS. 4(a) to 4(d) show examples of female master molds 300 with different hole patterns, suitable for implementing the method 100 of the present invention. FIG. 4(a) shows a master mold 300 with a square array of holes 320. FIG. 4(b) shows a master mold 300 with a hexagonal array of holes 320. FIG. 4(c) shows a master mold 300 with linear arrays of holes 320, and FIG. 4(d) shows a master mold 300 with a triangular array of holes 320. In the context of the present invention, the hole array of the female master mold 300 provides a base microneedle pattern that defines the possible locations at which microneedles can be formed to create the desired or customised microneedle array.

With reference again to FIG. 1, the general method 100 of producing a microneedle array comprises masking 110 one or more holes 320 in the master mold 300 at predefined locations in the hole array to create an array of unmasked holes corresponding to a desired microneedle pattern, and forming 120 a microneedle array having the desired microneedle pattern using the masked master mold. The method 100 can comprises a single masking step 110, or multiple masking steps 110 depending on whether the microneedle array is single or multi-cargo and how the cargo is introduced to the microneedles, as indicated by step 122 and explained below.

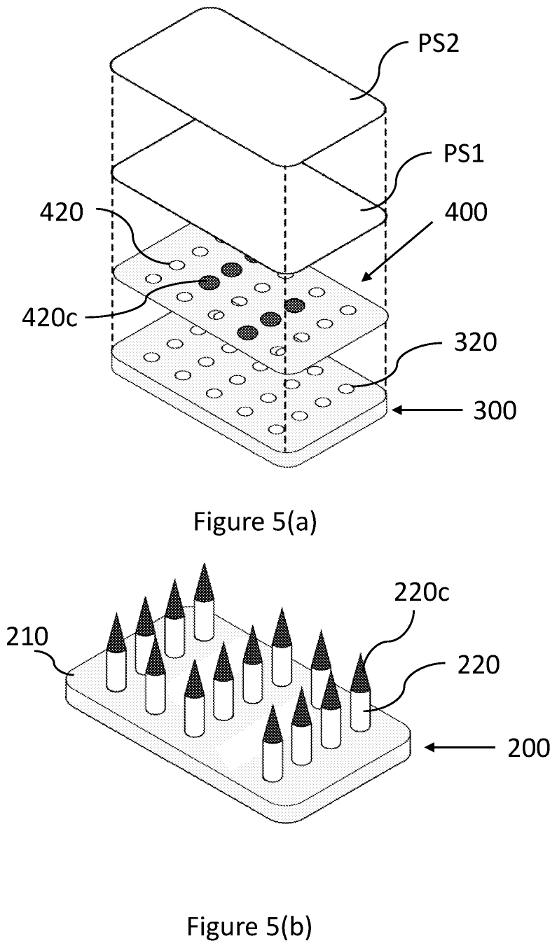

FIGS. 5(a) and 5(b) illustrate an example single masking step process for producing a microneedle array 200 in the desired microneedle pattern according to an embodiment of the method 100. The masking step 110 comprises aligning a masking layer 400 over the master mold 300 to cover the one or more holes 320 in the master mold 300 at the predefined locations. Once aligned, the masking layer 400 is attached or secured to the master mold 300, e.g. held in intimate contact with the master mold 300 place by compression, vacuum or van der Waals forces. The masking layer 400 is substantially planar and comprises an array of openings 420 at locations corresponding to the desired microneedle pattern. Aligning the masking layer 400 therefore comprises aligning the openings 420 of the masking layer 400 with holes 320 in the master mold 300. For example, this can be achieved using a mask aligner (a tool in which the masking layer 400 is translatable relative to the master mold 300 and comprises imaging means to precisely align the masking layer 400 to the master mold 300), optionally with the aid of alignment marks on the master mold 300 and masking layer 400, in a similar fashion to mask alignment performed in photolithography. The masking layer 400 can be aligned manually or automatically using the known techniques.

In step 120, a polymer solution PS1 containing a cargo 220c is cast into the masked master mold 300′. A vacuum and/or centrifuge can be applied to promote filling of the unmasked holes 320. All holes 320 of the master mold 300 that are not needed are therefore covered by the masking layer 400 and inaccessible during the casting step 120 resulting in a microneedle array 200 with the desired microneedle pattern, as shown in FIG. 5(b).

In step 130, a second polymer solution PS2 is cast over the first polymer solution PS1 to form a substrate 210 for the microneedle array 200. Residual polymer solution PS1 is removed prior to casting the second polymer solution PS2. In the single masking step process shown, the masking layer 400 can remain in place for the substrate casting step 130.

In step 140, the polymer solutions PS1, PS2 are cured/solidified and the microneedle array 200 is removed from the master mold 300 and masking layer 400, as shown in FIG. 5(b). Curing may comprise air drying, UV/light curing, baking or any other suitable method, depending in the specific polymer solution(s) used, as is known in the art.

In the single masking step process of FIG. 5(a) in which cargo 220c is contained in the polymer solution PS1, each microneedle 220 formed by the masking and casting step 110, 120 contains the same cargo 220c as shown. A multi-cargo microneedle array 200 can be produced by repeating the masking and casting steps 110, 120 multiple times with different masking layers 400 each forming a portion of the desired microneedle pattern, indicated in step 122 of FIG. 1 and described in more detail below.

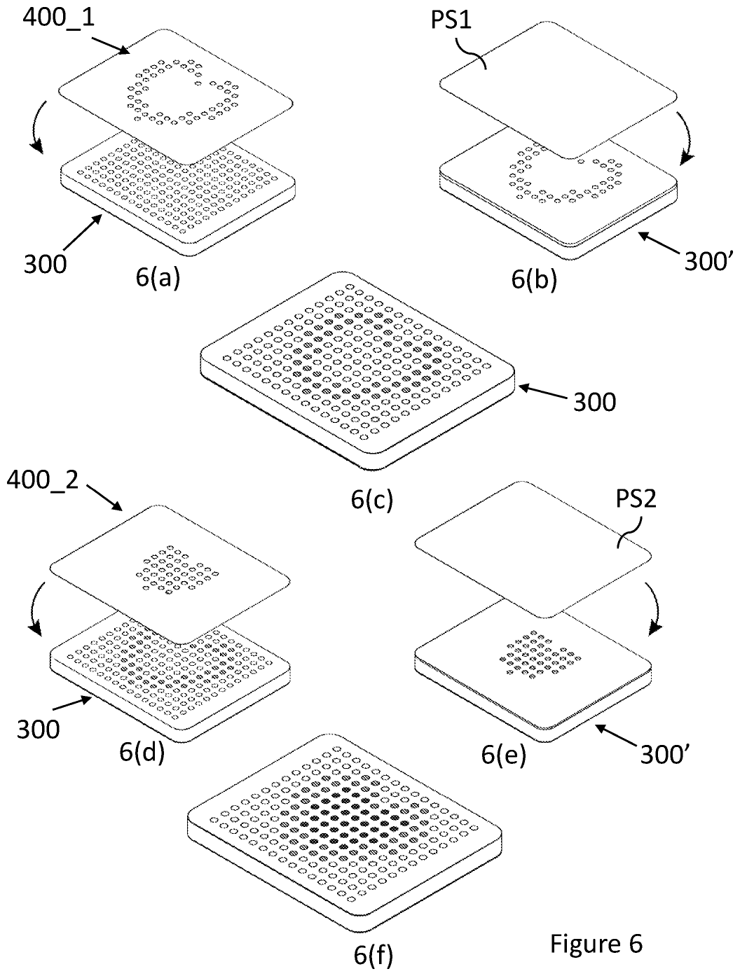

FIGS. 6(a)-6(j) illustrate a multi-masking step process for producing a multi-cargo microneedle array in a desired microneedle and cargo pattern. In this example, the desired needle pattern is a heart pattern comprising two different cargos. In step 110, a first masking layer 400_1 comprising a first array of openings 420 is aligned and attached to the master mold 300 to create a first array of unmasked holes corresponding to a first portion of the desired microneedle pattern, which in this case is an outline of a heart as shown in FIG. 6(a). In step 120, a first polymer solution PS1 containing a first cargo 220c is cast into the masked master mold 300′ as indicated in FIG. 6(b). A vacuum and/or centrifuge can be applied to promote filling of the unmasked holes 320. Residual polymer solution PS1 and the first masking layer 400_1 are then removed, as indicated in FIG. 6(c). Steps 110 and 120 are then repeated. A second masking layer 400_2 comprising a second array of openings 420 (different to the first array) is aligned and attached to the master mold 300 to create a second array of unmasked holes corresponding to a second portion of the desired microneedle pattern, which in this case forms the inner portion of the heart as shown in FIG. 6(d). Next, a second polymer solution PS1 containing a first cargo 220c is cast into the masked master mold 300′ as indicated in FIG. 6(e). A vacuum or centrifuge can be applied to promote filling of the unmasked holes 320. Residual polymer solution PS2 and the second masking layer 4002 are then removed, as indicated in FIG. 6(f). This multi-masking process can be repeated as many times as required for the desired needle and cargo pattern. In an embodiment, the base polymer of the first and second polymer solutions PS1, PS2 is the same, such that they differ only in the cargo 220c they contain. In another embodiment, the base polymer and the cargo 220c of each solution is different.

The process then proceeds to step 130 to form a substrate layer 210. This involves a final masking step, in which a third/final masking layer 400_3 comprising a third array of openings 420 is aligned and attached to the master mold 300 to create a third array of unmasked holes corresponding to the entire desired microneedle pattern, i.e. covering all holes that are not part of the desired microneedle pattern (heart), as shown in FIG. 6(g). The third array is the sum of the first and second arrays. A third polymer solution PS2 is then cast into the masked master mold 300′ as indicated in FIG. 6(h) to form a substrate 210 for the microneedle array 200. The substrate polymer PS3 does not contain cargo to be delivered, but may contain ink, e.g. for aesthetic purposes. Finally, in step 140, the polymer solutions PS1, PS2, PS3 are cured/solidified and the microneedle array 200 is removed from the master mold 300 as shown in FIG. 6(i). The masking layer 400_4 can remain in place or be removed. The microneedle patch 200 can then be packaged, e.g. in a sealed UV/light-fast bag.

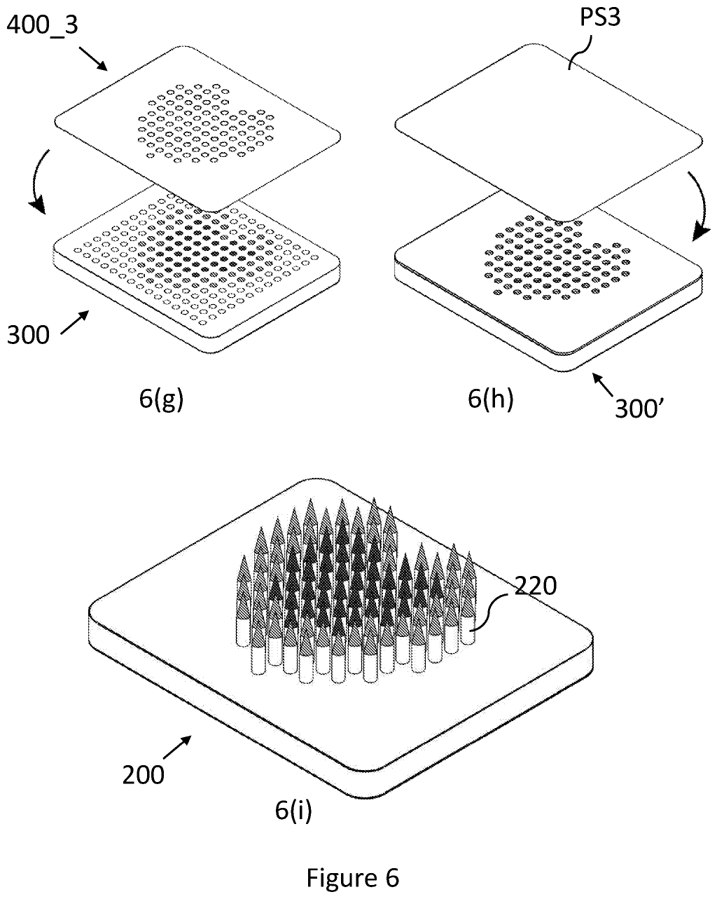

In another embodiment, cargo 220c or a polymer solution containing cargo 220c is deposited directly into holes 320 holes of the master mold 300 prior to casting the polymer solution PS1 (and optionally also prior to masking) by an inkjet printing process, as shown in FIGS. 7(a) to 7(f). In this case, the polymer solution PS1 does not need to contain a cargo 220c, but may optionally include a different cargo to the inkjet deposited cargo. Inkjet printing the cargo 220c allows multiple different cargos to be deposited into the array of holes in the same masking layer step to produce a multi-cargo microneedle array 200. Although inkjet printers are typically associated with printing inks, they can also be used to print polymers as well as other materials.

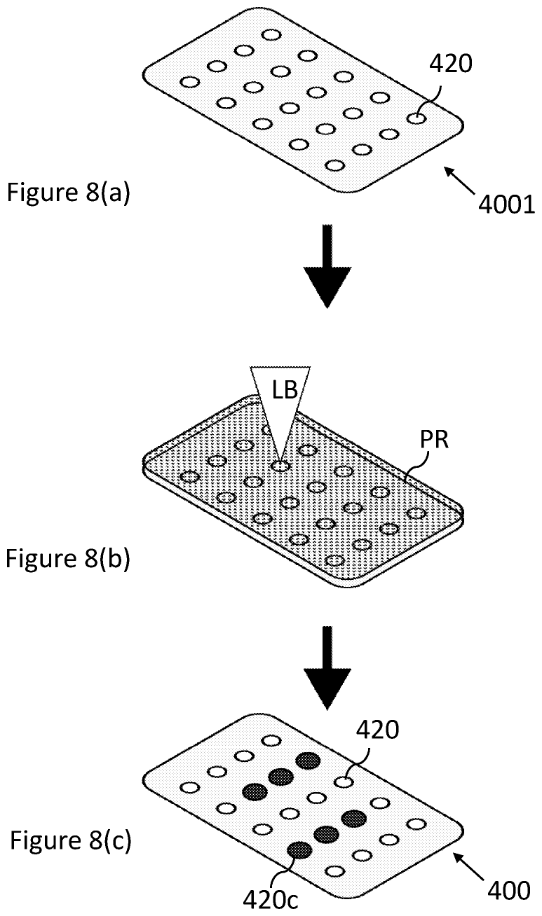

With reference to the example of FIGS. 7(a)-7(f), the desired needle pattern is again a heart pattern comprising two different cargos. In step 120, a first polymer solution PS1 containing a first cargo 220c1 is deposited directly into a first array of holes 320 corresponding to a first portion of the desired microneedle pattern, and a second polymer solution PS2 containing a second cargo 220c2 is deposited directly into a second array of holes 320 corresponding to a second portion of the desired microneedle pattern using an inkjet printer, as shown in FIGS. 7(a)-7(c). This step at least partially fills the unmasked holes 320 with polymer solution PS1, PS2. The inkjet printer nozzle deposits the polymer solutions PS1, PS2 at the specific locations of the holes 320 based on a microneedle image file or data file defining the locations of the microneedles, the microneedle cargo material, microneedle cargo, and relative amount of cargo. The microneedle data file is generated based on the geometry of the hole array and an input image/graphic, as will be described in more detail below with reference to FIG. 9.

Next, in step 110, a masking layer 400 comprising an array of openings 420 is aligned and attached to the master mold 300 to create an array of unmasked holes corresponding to the entire desired microneedle pattern, i.e. covering all holes that are not part of the desired microneedle pattern (heart), as shown in FIG. 7(d). Alternatively, the polymer solutions PS1, PS2 can be deposited into/through the unmasked holes 320 of the masked master mold 300′ (not shown). In step 130, a third polymer solution PS3 (not containing cargo) is then cast into/onto the masked master mold 300′ as indicated in FIG. 7(e) to form the substrate 210 for the microneedle array 200. Where the inkjet only partially fills the holes 320 with polymer solutions PS1, PS2, this step may fill the remainder of the holes and form the substrate 210. Finally, in step 140, the polymer solutions PS1, PS2, PS3 are cured/solidified and the microneedle array 200 is removed from the master mold 300 as shown in FIG. 7(f). The masking layer 400 can remain in place or be removed.

Where the inkjet deposits only cargo 220c1, 220c2 into the holes 320, step 130 may comprise casting a polymer solution PS1 (not containing cargo) into the masked master mold 300′ to form the microneedles and substrate 210 for the microneedle array 200.

In contrast to the master mold 300, the masking layer 400 is configured for the specific microneedle pattern. As such, the method 100 may include a step 108 of forming the masking layer 400 configured to cover the one or more holes 320 in the master mold 300 at the predefined locations.

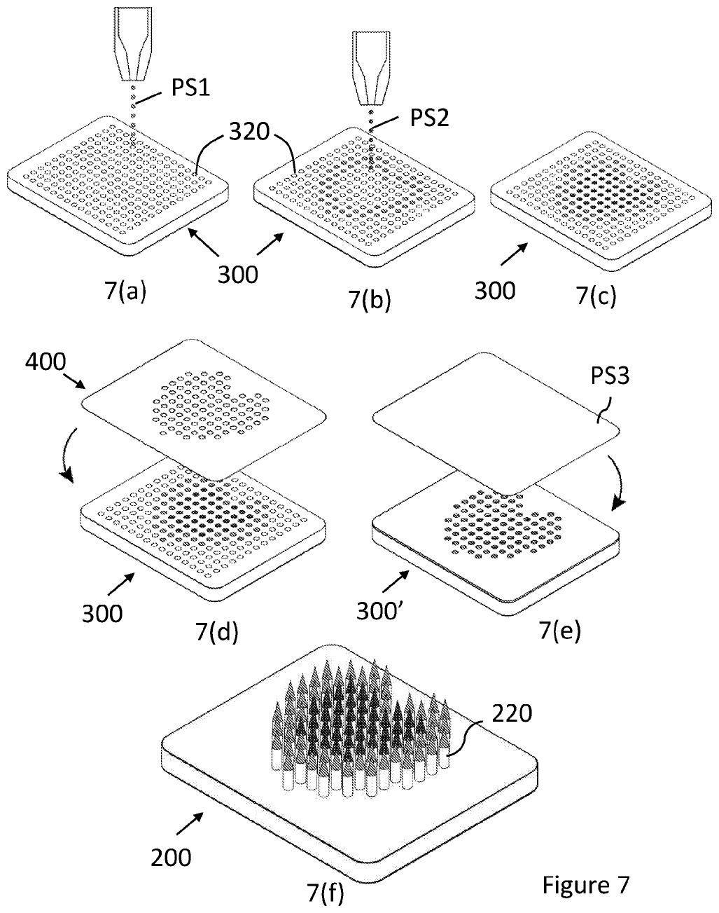

In an embodiment, the masking layer 400 is formed by closing one or more openings 420 in a standardised masking layer 4001 comprising an array of openings 420 that match the array of holes 320 in the master mold 300 to a create a customised masking layer 400 with an array of openings 420 corresponding to the desired microneedle pattern. FIGS. 8(a)-8(c) show an example process of selectively closing openings 420 in a standardised masking layer 4001. FIG. 8(a) shows an example of a standardised masking layer 400 with openings 420 matching that of the master mold 300. In FIG. 8(b), a layer of photo-polymer or photo-resist PR (e.g. SU-8 or other negative photoresists) is cast over the standardised masking layer 4001, and focused light or a focused laser beam LB is used to locally expose the photopolymer PR at locations of openings 420 that are not used in the desired microneedle pattern. This process of laser-induced photo-polymerisation causes the exposed photopolymer PR to cross-link or polymerize and become substantially insoluble to certain solvents. Unexposed areas of photopolymer PR can then be washed away, leaving the cross-linked photopolymer PR in place covering the openings at the required locations. The resulting customised masking layer 400 comprises a number of closed openings 420c and an array of openings 420 corresponding to the desired microneedle pattern, as shown in FIG. 8(c).

In another embodiment, the masking layer 400 can be formed by forming one or more openings 420 in a layer 400 at predefined locations corresponding to the desired microneedle pattern, e.g. by a photolithographic process or laser drilling (not shown).

In an embodiment, the masking layer(s) 400 are formed from or comprise a polymer, such as polystyrene, polylactic acid, polyethylene (PE), polyethylene terephthalate (PET), polypropylene (PP), or others, and have a thickness in the range 10 μm to 500 μm.

The desired microneedle pattern is determined based, at least in part, on an input digital pattern/image or computer graphic (e.g. the heart in FIGS. 6 and 7). The input computer graphic can be anything, e.g. including an image, shape, text and/or 2D encoded information. This input computer graphic is translated into a microneedle pattern that represents the graphical information. In an embodiment, the desired microneedle and cargo pattern is defined by a microneedle data file or image file, in which pixels of the image file correspond to one or multiple microneedles and contain information about the location, needle material, needle cargo and relative amount of cargo for each microneedle. This image file is used to create the customised masking layer(s) 400 and/or control the inkjet printer.

In an embodiment, the method 100 comprises a step 106 of translating an input image or computer graphic into microneedle data or a microneedle image file. The input computer graphic 10 is translated into a microneedle image file 30 based the geometry 20 of the array of holes 320 of the master mold 300 (i.e. the spacing/relative locations of holes 320 in the master mold 300).

FIG. 9 illustrates the process of translating an input computer graphic 10, in this case the heart shown in FIGS. 6 and 7, into a microneedle image file 30 that defines the microneedle and cargo pattern 40.

Step 106 comprises resampling and/or digitizing the input computer graphic 10 based on the geometry 20 of the array of holes to create an image file 30, in which pixels of the image file 30 corresponds to the locations of holes 320 in the master mold 300, as illustrated in FIG. 9. In principle, the input computer graphic can have any pixel resolution. Therefore, the resampling process (which in practice will typically comprise down-sampling the input graphic) allows any input graphic to be used. Step 106 further comprises assigning needle data to each pixel of the image file 30, such as needle or no needle, location, needle material, needle cargo and relative amount of cargo. In this way, the microneedle image file 30 defines a microneedle pattern 40 as shown in FIG. 9.

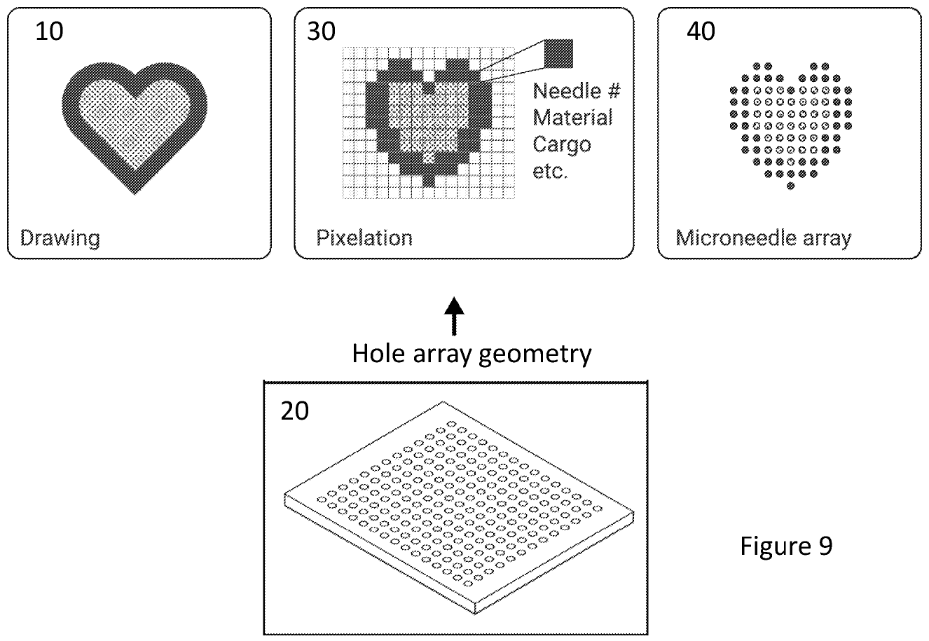

FIG. 10(a) shows an example of a microneedle array 200 configured as a multi-colour tattoo patch that can be produced according to the method 100. The patch comprises a patch substrate 210 with an adhesive portion 210a and microneedles 220 with different coloured ink cargos 220c1, 220c2, 220c3, 220c4 arranged in a specific pattern to convey graphical information, in this case the message “hello, world :-)”. FIG. 10(b) shows an example computer graphic stored in a microneedle image file 30 encoding the desired microneedle pattern 40. In this example, each pixel of the microneedle image file 30 corresponds to one microneedle 220 in the array 200 and contains information about the needle position, needle material, needle cargo and relative amount of cargo 220c, as described above. In other examples, one pixel may correspond to more than one microneedle 220 in the array 200.

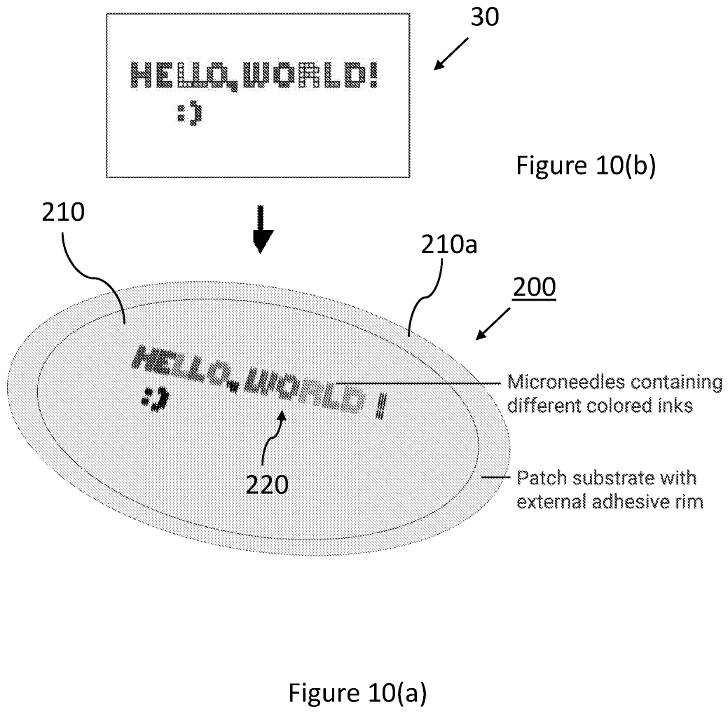

FIG. 11 shows a schematic block diagram of a method 500 of producing a microneedle array with a predefined needle and cargo pattern according to another embodiment of the invention.

In step 501, digital information in a digital input pattern/image is transformed into microneedle data defining a desired microneedle pattern, such that locations of microneedles in the microneedle pattern correspond to pixels in at least a portion of the digital input pattern or image. In step 502, a microneedle array is formed having the desired microneedle pattern. Step 502 may comprise forming the microneedle array using the molding process of FIG. 1 described above, or any other suitable process such as three-dimensional printing a male master mold according to the microneedle data. The key step is the translation of digital information in a digital input pattern/image into microneedle data.

In an embodiment, step 501 comprises generating a microneedle image file defining the properties of the microneedle array based on the digital input pattern/image and an array geometry, such that pixels of the microneedle image file correspond to the locations of microneedles (or groups/sub-arrays of microneedles) in the array. This may comprise resampling and/or digitizing the input computer graphic based on the geometry of the array. Each pixel of the microneedle image file contains microneedle information including one or more of: microneedle location, microneedle material, microneedle geometry, microneedle number, microneedle cargo, and relative amount of cargo. For example, each pixel can contain a binary value indicating the presence of a microneedle, and one or more additional values or data defining or encoding the properties of the respective microneedle at that location (e.g. cargo type, amount of cargo etc.).

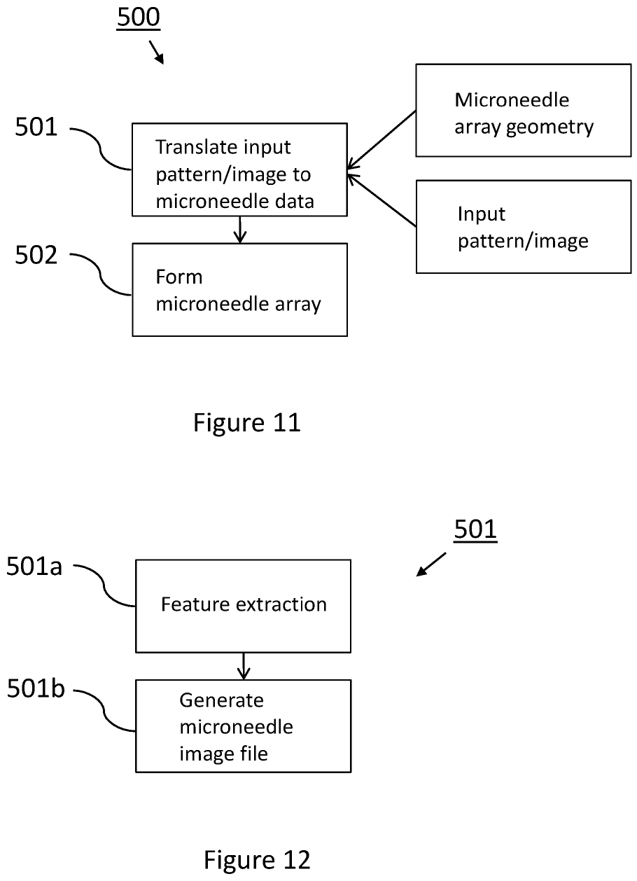

Step 501 may further comprise a step of detecting 501a one or more image features in the digital input pattern/image and generating 501b a microneedle image file/microneedle pattern based at least in part on the detected image feature, such that the area and/or perimeter of the microneedle pattern corresponds to the shape, size, and/or perimeter of the one or more image features, as illustrated in FIG. 12. In this context, an image feature is a certain region of the input image having certain properties, e.g. specific structures in the image such as points, edges or objects, or specific shapes defined in terms of curves or boundaries between different image regions or colours. Image features can be detected using computer vision or other image processing techniques known the art. Microneedle information such as the binary pixel value and ink colour can also be determined from the extracted image feature.

Where the cargo is ink or pigment, step 501b may comprise determining a colour of the ink or pigment for each microneedle in the microneedle pattern based on a colour of one or more pixels of the image feature, or based on the colour of one or more pixels in a region surrounding the image feature.

In this way, the microneedle array can be used to mask or camouflage the skin. For example, the digital input image may comprise an image of an area of skin or tissue including a pigmentation feature or a scar feature. The area and/or perimeter of the desired microneedle pattern can be defined to correspond to the size and shape of the pigmentation or scar feature, and the colour of the ink in the microneedle array can be defined to match the skin colour surrounding the skin feature so as to mask, camouflage or at least reduce the appearance/visibility of the skin feature.

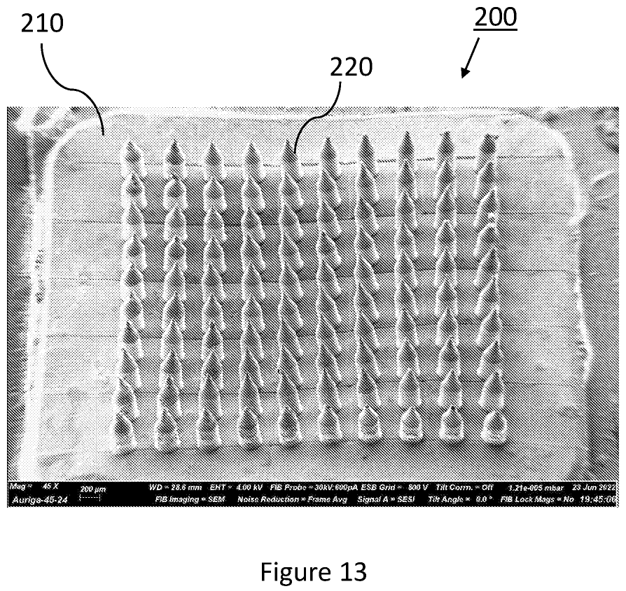

FIG. 13 shows a scanning electron micrograph of an example prototype tattoo patch 200 generating from digital microneedle data. The array is a 10×10 tattoo needle grid, whereby each microneedle 220 has a cylindrical base and conical tip. The microneedles are approximately 700 microns long with a diameter of 250 microns. The prototype array 200 was formed by 3D printing a male microneedle mold, making a female microneedle mold from the male array, casting a polymer solution into that female array and then removing the microneedle patch 200 after hardening of the polymer. The polymer solution used was 7.5% w/v 100 kDa hyaluronic acid and 5% w/v 3.4 kDa PEG containing tattoo ink.

As described above, the invention provides a way to produce microneedle arrays with specific microneedle patterns based on the translation of digital information in an input image into microneedle image file with pixels defining the properties of the array. Such a bespoke array has various applications.

Applications of a digitally generated microneedle array include but are not limited to: applying aesthetic or cosmetic (e.g. areolar reconstruction, eyebrow tattoos, lip tattoos, scalp micropigmentation, fingernail/anatomical re-pigmentation etc.) tattoos on the skin or tissue of a subject, camouflaging a skin feature (e.g. re-pigmentation of scars, stretchmarks, or areas of differing pigment that may be caused by sun exposure or vitiligo for example), applying unique identifiers to the skin such as machine-readable optical indicia encoding information, or applying biosensing tattoos.

In an embodiment, the method 100, 500 is used to produce a biosensing tattoo configured with a specific microneedle pattern to convey graphical information responsive to a change in body chemistry. In one example, the cargo 220c of at least some of the microneedles of the biosensing tattoo contain a biosensing material or ink that changes colour or visibility in responsive to a change in body chemistry, or temperature, or external factors such as temperature, light exposure, magnetic fields, or audio and electro-magnetic exposure and/or signals. Examples of detectable substances or parameter in the body include pH value, saccharides (e.g. glucose), ions (e.g. sodium, potassium, calcium, magnesium, chloride, bicarbonate), lipids, nucleic acids, and proteins. The microneedle pattern may be configured to provide an indication of presence or level of the sensed substance, e.g. glucose. The biosensing material may be comprise functionalised nano- or microparticles.

In an embodiment, the precision of prearranged needles and versatility of cargo provided by the method 100 can be used to produce a tattoo patch configured with a specific microneedle and cargo pattern to imprint encoded graphical information into the skin, e.g. a unique ID code or signature. The input digital image on which the microneedle pattern is based can comprise machine readable optical indicia such as a unique 2D code or pattern, barcode or quick response (QR) code. In this way, the tattoo patch can be used as a signature/authentication patch for tattoo artists. Such a tattoo patch can be used by a tattoo artist to give a seal of authentication to a piece of tattoo art. This “authentication patch” can contain invisible ink to the eye and only be readable by a camera device, such as a smart device. The “authentication patch” could be either a piece of graphical information or text with unique properties that could be compared to a database of images (e.g. input digital images used to produce the tattoo patches), or the authentication patch can comprise a machine readable 2D code that can only be deciphered by a computer device.

In another example application of microneedle patches formed using the method 100, 500, graphical information to be translated into a tattoo patch could be secured using a non-fungible token (NFT). A tattoo patch containing the visible graphical information could contain an additional invisible or visible 2D code that allows the tattoo owner to authenticate the tattoo as the physical token that belongs to the respective NFT.

In another example application, microneedle patches could be used to remove tattoos by using enzymes that degrade existing tattoo ink in the dermis by loading needles with respective enzyme cargos. In this example, an existing tattoo could be photographed. From the photograph one could extract the graphical information of the tattoo. This graphical information can then be translated into a microneedle and cargo pattern (defined by the microneedle image/data file) to create a tattoo patch that matches the existing tattoo. Enzymes or other cargos could then be efficiently and precisely delivered to the right locations on the skin without cargo being applied to unwanted locations in the skin. This concept of using a digital photograph to generate customised input graphical information for a microneedle and cargo pattern can also be applied to other applications where targeted delivery of cargo is required, such as wrinkle treatments or melanoma treatments.

The microneedle array can also be used to enable authentication of an existing tattoo of a subject. The microneedle array can be configured to deliver a unique identifier ID tattoo on the skin or tissue of the subject comprising a unique machine-readable optical indicia encoding authentication information. This could be placed next to the subject's existing tattoo e.g. an aesthetic tattoo drawn by a tattoo artist, and the artist's tattoo can then be authenticated by reading the information encoded in the ID tattoo, and comparing the authentication information to a database of identifiers associated with registered tattoo artists.

From reading the present disclosure, other variations and modifications will be apparent to the skilled person. Such variations and modifications may involve equivalent and other features which are already known in the art, and which may be used instead of, or in addition to, features already described herein.

Although the appended claims are directed to particular combinations of features, it should be understood that the scope of the disclosure of the present invention also includes any novel feature or any novel combination of features disclosed herein either explicitly or implicitly or any generalisation thereof, whether or not it relates to the same invention as presently claimed in any claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable sub-combination.

For the sake of completeness it is also stated that the term “comprising” does not exclude other elements or steps, the term “a” or “an” does not exclude a plurality, and any reference signs in the claims shall not be construed as limiting the scope of the claims.

1. A method of forming a microneedle array using a master microneedle mold with an array of holes for forming a microneedle array, comprising:
masking one or more holes in the master mold at predefined locations to create an array of unmasked holes corresponding to a desired microneedle pattern; and
forming a microneedle array having the desired microneedle pattern using the masked master mold,
wherein the desired microneedle pattern is based on at least a portion of a digital input pattern or image, and the locations of un-masked holes in the master mold correspond to pixels of the digital input pattern or image.

2. The method of claim 1, wherein masking one or more holes in the master mold comprises aligning a masking layer over the master mold to cover the one or more holes in the master mold at the predefined locations; and optionally or preferably, further comprising forming a masking layer configured to cover the one or more holes in the master mold at the predefined locations; and, optionally or preferably, wherein the masking layer comprises an array of openings corresponding to the locations holes in the master mold, and forming the masking layer comprises selectively closing one or more openings in the masking layer to create an array of openings corresponding to the desired microneedle pattern; and optionally or preferably, wherein the one or more openings are closed using a photolithography or laser-induced photo-polymerization process; or, wherein forming the masking layer comprises forming one or more openings in the masking layer at predefined locations corresponding to the desired microneedle pattern; optionally or preferably, by a photolithographic process, or laser or mechanical drilling process

3. (canceled)

4. (canceled)

5. (canceled)

6. The method of claim 1, further comprising:
determining a desired microneedle pattern based on a digital input pattern/image and the geometry of the array of holes of the master mold, such that the locations of un-masked holes in the master mold correspond to pixels of the digital input pattern or image; and/or
determining locations of one or more holes in the master mold to be masked based on a digital input pattern/image and the geometry of the array of holes of the master mold, such that the locations of un-masked holes in the master mold correspond to pixels of the digital input pattern or image.

7. The method of claim 1, wherein each pixel in the digital input pattern is represented by one or more microneedles in the desired microneedle pattern.

8. The method of claim 1, further comprising:
generating a microneedle image file defining the properties of the microneedle array based at least in part on a digital input pattern/image and the geometry of the array of holes of the master mold, wherein each pixel of the microneedle image file corresponds to one or more microneedles of the desired microneedle pattern; and masking one or more holes in the master mold at locations defined in the microneedle image file to create an array of unmasked holes corresponding to the desired microneedle pattern; and, optionally or preferably, wherein each pixel of the microneedle image file contains microneedle information including one or more of: microneedle location, microneedle material, microneedle geometry, microneedle number, microneedle cargo, and relative amount of cargo.

9. (canceled)

10. The method of claim 1, wherein forming the microneedle array comprises casting a polymer solution into the masked master mold; and, optionally or preferably, solidifying the polymer solution; and optionally or preferably, wherein the polymer solution contains a cargo and wherein the cargo comprises one or more of: an ink, a prophylactic drug, a therapeutic drug, a diagnostic or biosensing material, and a cosmetic material: and optionally or preferably, wherein forming the microneedle array further comprises depositing a polymer solution containing or encapsulating a cargo into the unmasked holes of the master mold prior to casting; optionally or preferably, by inkjet printing; and, optionally or preferably, wherein one or more microneedles in the microneedle array comprise a different cargo to at least one other microneedle in the microneedle array.

11. (canceled)

12. (canceled)

13. The method of claim 8, comprising depositing a cargo into unmasked holes of the master mold prior to casting based on information in the microneedle image file.

14. The method of claim 1, wherein:
the step of masking one or more holes in the master mold creates a first array of unmasked holes corresponding to a first portion of the desired microneedle pattern;
the step of forming the microneedle array forms a first portion of a microneedle array having the first portion of the desired microneedle pattern using the masked master mold and a polymer solution containing a first cargo; and the method further comprises:
masking one or more different holes in the master mold to create a second array of unmasked holes corresponding to a second portion of the desired microneedle pattern; and forming a second portion of the microneedle array having the second portion of the desired microneedle pattern using the re-masked master mold and a polymer solution containing a second cargo; or further comprising forming a substrate layer over the microneedle array; and, optionally or preferably, releasing the microneedle array from the masked master mold.

15. (canceled)

16. A method of forming a microneedle array, comprising:
transforming digital information in a digital input pattern/image into microneedle data defining a desired microneedle pattern, such that locations of microneedles in the microneedle pattern correspond to pixels in at least a portion of the digital input pattern or image; and
forming a microneedle array according to the desired microneedle pattern.

17. The method of claim 16, wherein forming the microneedle array comprises a molding process or a three-dimensional printing process.

18. The method of claim 16, wherein the step of transforming comprises:
generating a microneedle image file defining the properties of the microneedle array based at least in part on the digital input pattern/image, wherein each pixel of the microneedle image file corresponds to one or more microneedles of the desired microneedle pattern, and optionally forming the microneedle array based on information in the microneedle image file.

19. The method of claim 18, wherein each pixel of the microneedle image file contains microneedle information including one or more of: microneedle location, microneedle material, microneedle geometry, microneedle number, microneedle cargo, and relative amount of cargo; and/or wherein the step of generating a microneedle image file comprises: detecting an image feature in the digital input pattern/image; and generating a microneedle pattern based at least in part on the detected image feature, wherein the area and/or perimeter of the microneedle pattern corresponds to the shape, size, and/or perimeter of the image feature; and/or, wherein each microneedle contains a cargo to be delivered into the skin or tissue, and wherein the cargo is an ink or pigment, and wherein the step of generating a microneedle image file further comprises: determining a colour of the ink or pigment for each microneedle in the microneedle pattern based on a colour of one or more pixels of the image feature, or based on the colour of one or more pixels in a region surrounding the image feature, optionally to substantially match the colour of one or more pixels in or around the image feature.

20. (canceled)

21. (canceled)

22. The method of claim 18, wherein the digital input image is or comprises an image of an area of skin or tissue including a pigmentation feature or a scar feature, and the area and/or perimeter of the desired microneedle pattern corresponds to the size and shape of the pigmentation or scar feature; and optionally or preferably, comprising generating a microneedle image file defining a microneedle array configured to mask or camouflage a pigmentation feature or a scar feature in an area of skin or tissue.

23. (canceled)

24. A microneedle array for delivering cargo material into the skin or tissue, wherein the microneedle array comprises microneedles formed in a desired microneedle pattern, and
wherein the desired microneedle pattern is based on at least a portion of a digital input pattern or image, and the locations of the microneedles in the microneedle pattern correspond to pixels in the at least a portion of the digital input pattern or image.

25. The microneedle array of claim 24, wherein the microneedle pattern is configured to convey graphical information; and/or wherein the area and/or perimeter of the microneedle pattern corresponds to a size and/or shape of an image feature in the input image; and/or therein the digital input image is or comprises an image of an area of skin or tissue including a pigmentation feature or a scar feature, and the area and/or perimeter of the desired microneedle pattern corresponds to the size and shape of the pigmentation or scar feature.

26. (canceled)

27. (canceled)

28. The microneedle array of claim 24, wherein each microneedle contains a cargo to be delivered into the skin or tissue, and wherein the cargo is an ink or pigment, and wherein the colour of the ink or pigment for each microneedle in the microneedle pattern is based on a colour of one or more pixels of the image feature, or based on the colour of one or more pixels in a region surrounding the image feature, optionally to substantially match the colour of one or more pixels in or around the image feature.

29. The microneedle array of claim 24, wherein the digital input image is or comprises an image of an area of skin or tissue including a pigmentation feature or a scar feature, and wherein the microneedle array is configured to camouflage the pigmentation feature or scar feature.

30. The microneedle array of claim 24, comprising: one or more microneedles comprising a first cargo to be delivered; and one or more microneedles a second cargo to be delivered, the second cargo being different to the first cargo and/or wherein the microneedle array is formed by a molding process or a three-dimensional printing process; or wherein the microneedle array is formed by a method, comprising: masking or more holes in a master mold at predefined locations to create an array of unmasked holes corresponding to a desired microneedle pattern; and forming a microneedle array having the desired microneedle pattern using the masked master mold, wherein the desired microneedle pattern is based on at least a portion of a digital input pattern or image, and the locations of unmasked holes in the master mold correspond to pixels of the digital input patter or image; and or wherein the microneedle array defines a needle pattern configured to convey graphical information, and the one or more microneedles containing or encapsulating the first cargo form a first portion of the needle pattern, and the one or more microneedles containing or encapsulating the second cargo form a second portion of the needle pattern.

31. (canceled)

32. (canceled)

33. The microneedle array of claim 24, wherein the microneedle array is configured to deliver a biosensing tattoo that conveys graphical information and changes colour or visibility in response to a change in body chemistry; and, optionally preferably, wherein the first cargo comprises an ink, and the second cargo comprises a bio-sensing material configured to change colour or visibility in response to an interaction with a chemical substance and/or wherein the microneedles contain a cargo to be delivered into the skin; and wherein the cargo comprises one or more of: an ink, a prophylactic drug, a therapeutic drug, a diagnostic or biosensing material, and a cosmetic material.

34. (canceled)

35. The microneedle array of claim 24 to camouflage a skin pigment or scar feature on the skin or tissue of a subject; and/or apply a machine-readable optical indicia encoding information on the skin or tissue of a subject; and or apply a tattoo on the skin or tissue of a subject; and or authenticate a first tattoo of a subject, wherein the microneedle array is configured to deliver a second tattoo on the skin or tissue of the subject having the first tattoo comprising a machine-readable optical indicia encoding authentication information, and wherein the first tattoo is authenticated by reading the authentication information encoded in the second tattoo; and comparing the authentication information to a database of identifiers associated with registered tattoo artists

36. (canceled)

37. (canceled)

38. (canceled)
